(19) United States
(12) Patent Application Publication
(10) Pub. No.: US 2014/0011197 A1
(43) Pub. Date: Jan. 9, 2014

(54) Alzheimer's Disease Cellular Model for Diagnostic and Therapeutic Development

(71) Applicant: The Regents of the University of California, Oakland, CA (US)

(72) Inventors: Lawrence Goldstein, La Jolla, CA (US); Mason Israel, La Jolla, CA (US)

(73) Assignee: The Regents of the University of California, Oakland, CA (US)

(21) Appl. No.: 13/967,682

(22) Filed: Aug. 15, 2013

Related U.S. Application Data
(63) Continuation of application No. PCT/US2012/025354, filed on Feb. 16, 2012.
(60) Provisional application No. 61/443,311, filed on Feb. 16, 2011.

(51) Int. Cl. G01N 33/50; C12Q 1/68; G01N 33/68; C12N 5/0793; G01N 33/487

(52) U.S. Cl. CPC G01N 33/5058; C12N 5/0619; G01N 33/48728; G01N 33/6896; C12Q 1/6883; G01N 33/5091
USPC 435/6.11; 435/368; 435/456; 435/29; 435/7.4; 435/6.12; 435/6.13

(57) ABSTRACT

Stem-cell derived human neuronal models that mimic human Alzheimer's disease, including hereditary and sporadic Alzheimer's disease, comprising neural stem cells derived from human induced pluripotent stem cells. Also provided are purified human neurons developed from the neural stem cells that carry genomes from the Alzheimer's disease patients. The human neuronal models are neuronal models for hereditary and sporadic Alzheimer's disease, and are suitable for measurement of key behaviors of the Alzheimer's disease, providing further diagnostic tools for the development of sporadic Alzheimer's disease, and assisting in drug testing for the therapeutic treatment of Alzheimer's disease.

### CROSS REFERENCE TO RELATED APPLICATIONS

This application is a continuation of PCT Application No. PCT/US2012/025354 filed on Feb. 16, 2012, which claims priority to U.S. Provisional Application Ser. No. 61/443,311, filed Feb. 16, 2011, the entire contents of which are incorporated by reference herein.

### FIELD OF THE INVENTION

The present invention relates to the stem cell reprogramming technology for producing a human neuronal model for diagnosis and therapeutic treatment of Alzheimer's disease.

### BACKGROUND OF THE INVENTION

Alzheimer's disease (AD) is a common neurodegenerative disorder, defined post-ortem by the increased presence of amyloid plaques and neurofibrillary tangles (NFTs) in the brain^{3,4}. Amyloid plaques are extracellular deposits consisting primarily of amyloid-β (Aβ) peptides, and NFTs are intraneuronal aggregations of hyperphosphorylated tau, a microtubule-associated protein involved in microtubule stabilization⁵. The causative relationship between amyloid plaque/AB and tau pathologies is unclear in humans. Although, the vast majority of Alzheimer's disease is apparently sporadic with significant non-Mendelian genetic contributions⁶, analysis of cellular and animal models of rare, dominantly inherited, familial forms of Alzheimer's disease (fAD) have driven most ideas about disease mechanisms. These rare cases have mutations or a duplication of APP, which encodes the Amyloid Precursor Protein, or mutations in the presenilin genes, which encode proteolytic enzymes that cleave APP into AB and other fragments.

Mouse models that overexpress fAD mutations develop extensive plaque deposition and amyloid-associated pathology, but NFTs and significant neuronal loss are conspicuously absent^{7,8}. Fetal human cortical cultures have also been used to study the APP-tau relationship. For example, cortical cultures treated with 20 μM AB have elevated pTau⁹.

However, it is still unclear whether physiologically relevant levels of AB directly cause elevated pTau and what kinases are directly involved in this aberrant phosphorylation. Additionally, experimental approaches using fetal human neurons are hindered by limited availability of samples and unknown genetic backgrounds. The recent development of induced pluripotent stem cells (iPSC)¹⁰ has allowed investigation of phenotypes of dominantly inherited disease in vitro¹¹⁻¹³. However, not all diseases have been successfully modeled using iPSC¹⁴, and it is unclear whether iPSC can be used as tools to study sporadic forms of disease.

The understanding of Alzheimer's disease pathogenesis is currently limited by difficulties in obtaining live neurons from patients and the inability to model the sporadic form of Alzheimer's disease. Researchers have studied sampling of cerebrospinal fluid and analysis for Alzheimer's disease, and measured behavior of fibroblasts and other non-neuronal cell types from people with sporadic Alzheimer's disease. Animal models of Alzheimer's disease have been developed, however, these animal models do not completely mimic true human disease, and none of these animal models are neuronal models of the disease.

Therefore, there is a need to develop a human neuronal model that more accurately mimics true human Alzheimer's disease, and then use of such model for diagnosing and treating Alzhimer's disease.

### SUMMARY OF THE INVENTION

The present invention provides stem-cell derived human neuronal models that mimic human Alzheimer's disease, including hereditary and sporadic Alzheimer's disease. In certain embodiments, the stem-cell derived human neuronal models comprises human neural cells derived from human induced pluripotent stem cells (iPSCs). The present invention further provides purified human neurons developed from neural precursor cells and carrying genomes from the Alzheimer's disease patients. The purified human neurons present key indicators of Alzheimer's disease, including, but not limited to, measurements of proteolytic processing of one or more amyloid precursor proteins, phosphorylation of tau protein, activation of key kinase GSK3, measurement of synaptic phenotype, autphagy, and other disease behaviors.

The present invention also provides a method of making human neuronal models. In certain embodiments, the invention method comprises some or all of the following steps: a) isolating fibroblasts, or other cells, from a patient with Alzheimer's disease; b) reprogramming said fibroblasts, or other cells, to induced pluripotent stem cells (iPSCs); c) further differentiating said iPSCs into cultures containing neural rosettes; d) purifying neural precursor cells (NPCs); e) further differentiating purified NPCs into heterogeneous cultures containing neurons; f) purifying neurons from the heterogeneous cultures; and g) further culturing purified neurons into a substantially homogeneous neural culture containing at least 90% neurons. In certain embodiments, the iPSCs are reprogrammed by transducting the fibroblasts with retrovirus encoding OCT4, SOX2, KLF4, c-MYC, optionally with EGFP. Well-known or later developed methods of isolating, reprogramming, differntiating, purifying, and culturing the cells are also contemplated within the scope of the prevent invention.

The substantially homogeneous neural culture produced by the present invention is characterized by neural markers, including, but not limited to, glutamatergic, cholingeric, or GABAergic markers, such as VGLUT1, CHAT and GAD67, respectively. In certain embodiments, glia can be added to the neural culture produced by the present invention. Furthermore, the purified neurons from the homogeneous neural culture are characterized for action potentials and spontaneous currents, and further measured for key characteristics of Alzheimer's disease, including, but not limited to measurements of proteolytic processing of one or more amyloid precursor protein, phosphorylation of tau protein, activation of GSk3 kinase, measurement of synaptic phenotype, autophagy, or other disease behaviors.

Furthermore, the present invention provides a method for diagnosing, prognosing and predicting a likelihood of development of Alzheimer's disease, and particularly for predicting a sporadic Alzheimer's disease, using the invention human neuronal model for measuring the key behaviors from the purified neurons carrying the genome of the Alzheimer's disease patients. Moreover, the present invention also provides a method of screening and testing a candidate drug for Alzheimer's disease treatment using the human neuronal model of the present invention.

The invention also provides a method of identifying a candidate therapeutic agent for Alzheimer's disease treatment comprising combining the human neuronal model with an agent, observing therapeutically beneficial changes in the expression profile, phenotype or morphology of the neuronal model in response to the combination with the agent, thereby identifying the agent as a candidate therapeutic agent.

Therefore, the human neuronal models of the present invention are the first true human neuronal model for hereditary and sporadic Alzheimer's disease, and are suitable for measurement of key behaviors of the Alzheimer's disease, providing further diagnostic tools for the development of sporadic Alzheimer's disease, and as an important research tool for assisting in drug testing for the therapeutic treatment of Alzheimer's disease.

### BRIEF DESCRIPTION OF THE DRAWINGS

FIG. 1. Properties of patients and their fibroblasts. (a) Summary of patient information. MMSE, Mini Mental State Examination (max. score=30). (b) APP^{Dp}1 and 2 fibroblasts secrete increased levels of Aβ¹⁻⁴⁰ compared to NDC cells (P=0.01 and 0.0008, respectively, n=3). (c) No significant difference in Aβ 1-42/1-40 or 1-38/1-40 between patients.

FIG. 2. Generation of iPSC lines and purified neurons from APP^{Dp}, sAD, and NDC fibroblasts. (a-b) iPSC lines express nanog and TRA1-81. (c-d) iPSC-derived, FACS-purified NPCs express SOX2 and nestin. (e-h) iPSC-derived, FACS-purified neurons express MAP2 and βIII-tubulin. (i) Representative action potentials in response to somatic current injections. (j-k) Between individuals, no significant difference was found in ability of iPSC to generate NPCs at day 11 (P=0.32), or in the ability of NPCs to form neurons at 3 weeks (P=0.17). All images are representative. Scale bars, 50 μm.

FIG. 3. Increased Aβ, phospho-tau, and active GSK3β in APP^{Dp} and sAD2 neuronal cultures. (a) Neurons from APP^{Dp}1, APP^{Dp}2, and sAD2 secrete increased Aβ¹⁻⁴⁰ compared to NDC samples (P<0.05, 0.0001 and 0.05, respectively). (b) The difference in Aβ¹⁻⁴⁰ levels between APP^{Dp}1, APP^{Dp}2 and sAD2 samples versus NDC samples is larger in neurons versus fibroblasts. (c) Neurons from APP^{Dp}1, APP^{Dp}2, and sAD2 have increased pTau^{Thr231}/tTau compared to NDC samples (P=0.0006, 0.001 and 0.01, respectively). (d) Neurons from APP^{Dp}1, APP^{Dp}2, and sAD2 have elevated aGSK3β (% non-phospho-Ser9) compared to NDC samples (P<0.001, 0.0001 and 0.0001).

FIG. 4. The relationship between Aβ, pTau, and aGSK3β in iPSC-derived neurons. (a-b) Aβ¹⁻⁴⁰ levels strongly correlate with both pTau and aGSK3β levels (r=0.91 and r=0.92, respectively). (c) aGSK3β and pTau also strongly correlate (r=0.83). (d) Treatment of APP^{Dp}2 neurons for 24 hours with γ- or β-secretase inhibitors significantly reduced Aβ¹⁻⁴⁰ levels (P<0.0001). (e-f) β- but not γ-secretase inhibitors significantly reduced pTau (P=0.02 and 0.02) and aGSK3β levels (P=0.01 and 0.0001).

FIG. 5. Summary of main results. Primary cell cultures from 2 non-demented controls (NDC1, 2) 2 sporadic AD patients (sAD1, 2), and 2 familial AD patients (APP^{Dp}1, 2) were reprogrammed into patient-specific iPSC lines. Neurons were generated from iPSC lines by directed differentiation and FACS purification. Purified neurons from patients sAD2, APP^{Dp}1 and APP^{Dp}2 had significantly higher levels of secreted Aβ¹⁻⁴⁰, phospho-Tau and active GSK3β relative to NDC neurons. Inhibition of β-secretase with small molecules caused a significant reduction in the levels of Aβ¹⁻⁴⁰, phospho-Tau and active GSK3β.

FIG. 6. Additional characterization of patient fibroblasts. FIG. 6a is a representative brightfield image of fibroblast cultures (line NDC1 shown). FIG. 6b is familial AD samples contained 3 copies of the APP locus, while other samples were diploid. Down's, Down's syndrome fibroblasts. FIG. 6c is familial AD fibroblasts expressed higher levels of APP mRNA relative to NDC and sAD samples.

FIG. 7. Additional characterization of iPSC lines. FIG. 7a is a representative brightfield image of an iPSC line co-cultured on MEFs (line NDC1.2 shown). Note hESC-line morphology. FIGS. 7b-7e are all iPSC lines expressed SOX2 derived from the endogenous locus, formed embryoid bodies that contained cells indicative of endodermal and mesodermal germ layers, and maintained euploid karyotypes (representative data shown). AFP, α-fetoprotein (endodermal); SMA, a smooth-muscle actin (mesodermal). FIG. 7f is a representative brightfield image of an iPSC line differentiated on PA6 stromal cells for 11 days. Many neural rosette-like structures were present in cultures at this timepoint. FIG. 7g is a brightfield image of NPCs differentiated for 3 weeks. Note difference in homogeneity with FACS purified neurons (FIG. 2i). FIG. 7h is neuronal markers and phosphorylated tau were detected in cultures of NPCs differentiated for 3 weeks. Note the absence of tau in fibroblasts. FIG. 7i is an APP copy number was correctly maintained in iPSC-derived NPCs differentiated for 3 weeks.

FIG. 8. Transgene silencing in iPSCs. FIG. 8a is a transgene RNA expression levels in undifferentiated iPSCs relative to fibroblasts 3 days after retrovival transduction (“Td fibro”). Primers detected a tag common to all transgenes. FIG. 8b is the percentage of EGFP⁺ cells in iPSCs, differentiated NPCs (dNPCs) and their parental transduced fibroblasts.

FIG. 9. Chart summarizing the differentiation method.

FIG. 10. RNA levels of neuronal subtype markers. QPCR was performed on purified neurons with primers specific to VGLUT1, CHAT, and GAD67 (glutamatergic, cholinergic and gabaergic markers, respectively) and normalized to levels of the average of two housekeeping genes (TBP and NONO). FIGS. 10a-10c are expression levels per iPSC line. FIGS. 10d-10f, when grouped by individual, none of the patients samples were significantly different than NDC samples (P>0.05). Correlation coefficients with AB, pTau/tTau and aGSK3β listed in Table 2b.

FIG. 11. Additional electrophysiological properties of purified neurons. FIG. 11a is normal transient sodium and sustained potassium currents in response to voltage step depolarizations. FIG. 11b is spontaneous currents resulting from synaptic activity when voltage-clamped at −70 mV. 13 of 13 neurons analyzed exhibited currents and action potentials when current clamped and voltage claimed. 1 of 13 neurons analyzed exhibited spontaneous synaptic currents.

FIG. 12. Levels of Aβ¹⁻⁴⁰ (a), pTau/tTau (b), and aGSK3β (c) categorized by iPSC line.

FIG. 13. Effect of β- and γ-secretase inhibitors per line.

### DETAILED DESCRIPTION OF THE INVENTION

The present invention provides stem cell-derived human neuronal models that mimic human Alzheimer's disease, including hereditary and sporadic Alzheimer's disease. The present invention also provides methods of making the invention human neuronal models and methods of using such human neuronal models for diagnosing, prognosing, predicting a likelihood of development of Alzheimer's disease, and further screening and testing a candidate drug for the treatment of Alzheimer's disease.

Unless otherwise noted, the terms used herein are to be understood according to conventional usage by those of ordinary skill in the relevant art. In addition to the definitions of terms provided below, definitions of common terms in molecular biology may also be found in Rieger et al., 1991 Glossary of genetics: classical and molecular, 5th ed, Berlin: Springer-Verlag; in Current Protocols in Molecular Biology, F. M. Ausubel et al., eds., Current Protocols, a joint venture between Greene Publishing Associates, Inc. and John Wiley & Sons, Inc., (1998 Supplement); in Current Protocols in Cell Biology, J. S. Bonifacino et al., eds., Current Protocols, John Wiley & Sons, Inc. (1999 Supplement); and in Current Protocols in Neuroscience, J. Crawley et al., eds., Current Protocols, John Wiley & Sons, Inc. (1999 Supplement). It is to be understood that as used in the specification and in the claims, “a” or “an” can mean one or more, depending upon the context in which it is used. Thus, for example, reference to “a cell” can mean that at least one cell can be utilized.

In certain embodiments, the present invention provides human neuronal models comprising derivation and neuronal differentiation of induced pluripotent stem cells (iPSC) from patients with familial form of Alzheimer's disease (fAD) and sporadic Alzheimer's disease (sAD), as well as from non-demented, age-matched controls. More specifically, the present invention provides human neuronal models comprising human induced pluripotent stem cells (iPSCs) reprogrammed from fibroblasts obtained from control, sporadic, or hereditary Alzheimer's disease patients. The iPSCs are further converted into neural stem cells, which are then differentiated into neural culture containing human neurons. The derived human neurons can be purified and further used for measurement of key behaviors including, but not limited to, measurements of proteolytic processing of the amyloid precursor protein, phosphorylation of the tau protein, and activation of a key kinase GSK3. The iPSC-derived human neural stem cells are also suitable for measurement of synaptic phenotype, autophagy, and other disease behaviors.

The present invention also provides a method of making human neuronal models. In certain embodiments, the invention method comprises one or more of the following steps: a) isolating cells, such as fibroblasts from a patient with Alzheimer's disease; b) reprogramming said cells, such as fibroblasts to induced pluripotent stem cells (iPSCs); c) further differentiating said iPSCs into cultures containing neural rosettes; d) purifying neural precursor cells (NPCs); e) further differentiating purified NPCs into heterogeneous cultures containing neurons; f) purifying neurons from the heterogeneous cultures; and g) further culturing purified neurons into a homogeneous neural culture containing at least 90% neurons. In certain embodiments, the iPSCs are reprogrammed by transducting the fibroblasts with retrovirus encoding OCT4, SOX2, KLF4, c-MYC, optionally with EGFP. Other known or later developed methods of making iPSCs are also contemplated within the scope of the prevent invention.

As used herein, the present invention contemplates any stem cells including pluripotent stem cells, induced pluripotent stem cells (iPSCs) derived from non-pluripotent cells (e.g., fibroblasts), multipotent stem cells, totipotent stem cells, embryonic stem (ES) cells, and stem cells derived from fetal and adult tissues. In certain embodiments, the present invention provides making induced pluripotent stem cells (iPSCs) derived from fibroblasts from a patient having Alzheimer's disease.

As used herein, the term “pluripotent” refers to a cell capable of at least developing into one of ectodermal, endodermal and mesodermal cells. In one preferred embodiment, the term “pluripotent” refers to cells that are totipotent and multipotent. As used herein, the term “totipotent cell” refers to a cell capable of developing into all lineages of cells. As used herein, the term “multipotent” refers to a cell that is not terminally differentiated. In one preferred embodiment the pluripotent cell is a neural precursor cell and the pluripotent cell culture is a neural precursor cell culture. The pluripotent cells of the present invention can be derived from any stem cells or non-pluripotent cells, such as fibroblasts, of the patient of interest, i.e., over exhibiting or having a potential disposition for Alzheimer's Disease using any method known to those of skill in the art at the present time or later discovered. For example, the pluripotent stem cells can be produced using induction, de-differentiation and nuclear transfer methods which are known in the art or later developed. Stem cells may be generated in adherent culture or as cell aggregates in suspension culture in the presence or absence of one or more bioactive components or factors.

As used herein, the terms “bioactive component” and “bioactive factor” refer to any compound or molecule that induces a pluripotent cell to follow a differentiation pathway toward a neural cell. Alternatively, the bioactive component may act as a mitogen or as a stabilizing or survival factor for a cell differentiating towards a neural cell. While the bioactive component may be as described below, the term is not limited thereto. The term “bioactive component” as used herein includes within its scope a natural or synthetic molecule or molecules which exhibit(s) similar biological activity.

The present invention provides induced pluripotent stem cells (iPSCs) derived from fibroblasts, or other cells, from a patient of Alzheimer's disease, such iPSCs are further differentiated to neuronal stem cells, or neuronal precursors, in a cell differentiation environment to a desired degree. As used herein, the term “cell differentiation environment” refers to a cell culture condition wherein the stem cells are induced to differentiate into neural progenitor cells, or are induced to become a cell culture enriched in neural cells. Preferably the neural progenitor cell lineage induced by the growth factor will be homogeneous in nature. The term “homogeneous,” refers to a population that contains more than 80%, 85%, 86%, 87%, 88%, 89%, 90%, 91%, 92%, 93%, 94%, 95%, 96%, 97%, 98%, or 99% of the desired neural cell lineage.

In one embodiment, the cell differentiation environment comprises a cell culture condition suitable for cell differentiation and growing. In certain embodiments, the cell differentiation environment is a suspension culture whereby cells are not tightly attached to a solid surface when they are cultured. Non-limiting examples of suspension cultures include agarose suspension cultures, and hanging drop suspension cultures. In other embodiments, the cell differentiation environment can also contain supplements such as L-Glutamine, NEAA (non-essential amino acids), P/S (penicillin/streptomycin), N2 supplement (5 μg/ml insulin, 100 μg/ml transferrin, 20 nM progesterone, 30 nM selenium, 100 μM putrescine (Bottenstein, and Sato, 1979 PNAS 76, 514-517) and β-mercaptoethanol (β-ME).

It is contemplated that additional factors may be added to the cell differentiation environment, including, but not limited to fibronectin, laminin, heparin, heparin sulfate, retinoic acid, members of the epidermal growth factor family (EGFs), members of the fibroblast growth factor family (FGFs) including FGF2 and/or FGF8, members of the platelet derived growth factor family (PDGFs), transforming growth factor (TGF)/bone morphogenetic protein (BMP)/growth and differentiation factor (GDF) family antagonists including but not limited to noggin, follistatin, chordin, gremlin, cerberus/DAN family proteins, ventropin, and amnionless. TGF, BMP, and GDF antagonists could also be added in the form of TGF, BMP, and GDF receptor-Fc chimeras. Other growth factors may include members of the insulin like growth factor family (IGF), the wingless related (WNT) factor family, and the hedgehog factor family.

Additional factors may be added to promote neural stem/progenitor proliferation and survival as well as neuron survival and differentiation. These neurotrophic factors include, but are not limited to, nerve growth factor (NGF), brain derived neurotrophic factor (BDNF), neurotrophin-3 (NT-3), neurotrophin-4/5 (NT-4/5), interleukin-6 (IL-6), ciliary neurotrophic factor (CNTF), leukemia inhibitory factor (LIF), cardiotrophin, members of the transforming growth factor (TGF)/bone morphogenetic protein (BMP)/growth and differentiation factor (GDF) family, the glial derived neurotrophic factor (GDNF) family including, but not limited to, neurturin, neublastin/artemin, and persephin and factors related to and including hepatocyte growth factor. Neural cultures that are terminally differentiated to form post-mitotic neurons may also contain a mitotic inhibitor or mixture of mitotic inhibitors including, but not limited to, 5-fluoro 2′-deoxyuridine and cytosine β-D-arabino-furanoside (Ara-C). In another embodiment, the cell differentiation environment can comprise compounds that make the neural cells more resistant to apoptosis.

In other embodiments, the cell differentiation environment comprises an adherent culture. As used herein, the term “adherent culture” refers to a cell culture system whereby cells are cultured on a solid surface, which may in turn be coated with a substrate. The cells may or may not tightly adhere to the solid surface or to the substrate. The substrate for the adherent culture may further comprise any one or combination of polyornithine, laminin, poly-lysine, purified collagen, gelatin, extracellular matrix, fibronectin, tenacin, vitronectin, poly glycolytic acid (PGA), poly lactic acid (PLA), poly lactic-glycolic acid (PLGA) and feeder cell layers such as, but not limited to, primary astrocytes, astrocyte cell lines, glial cell lines, bone marrow stromal cells, primary fibroblasts or fibroblast cells lines. In addition, primary astrocyte/glial cells or cell lines derived from particular regions of the developing or adult brain or spinal cord including, but not limited to, olfactory bulb, neocortex, hippocampus, basal telencephalon/striatum, midbrain/mesencephalon, substantia nigra, cerebellum or hindbrain may be used to enhance the development of specific neural cell sub-lineages and neural phenotypes.

As used herein, the term “neurons” or “neural cell” can be used interchangeably, including, but not limited to, a glial cell; a neural cell of the central nervous system, such as a dopaminergic cell, a differentiated or undifferentiated astrocyte or oligodendrocyte; a neural progenitor, a glial progenitor, an oligodendrocyte progenitor, and a neural cell of the peripheral nervous system. “Neural cell” as used in the context of the present invention, is meant that the cell is at least more differentiated towards a neural cell type than the pluripotent cell from which it is derived. Also as used herein, producing a neural cell encompasses the production of a cell culture that is enriched for neural cells. In preferred embodiments, the term “enriched” or “substantially homogeneous” refers to a cell culture that contains more than approximately 50%, 55%, 60%, 65%, 70%, 75%, 80%, 85%, 90%, or 95% of the desired cell lineage.

In certain embodiments, the present invention provides that primary fibroblasts from patients with familial AD (caused by a duplication of APP^{1,2}, APP^{Dp}), sporadic AD (sAD), and non-demented control individuals (NDCs) were reprogrammed into iPSC lines. In certain embodiments, the iPSCs are reprogrammed by transducting the fibroblasts with retrovirus encoding OCT4, SOX2, KLF4, c-MYC, optionally with EGFP. Methods for making or reprogramming into iPSCs from fibroblasts are known in the art, and the present invention contemplates all the currently available and further developed methods including any improvements of making or reprogramming into iPSCs.

The present invention further provides differentiating the reprogrammed iPSCs into cultures containing neural rosettes, purifying and further differentiating neural precursor cells (NPCs) of the neural rosettes into heterogeneous cultures containing neurons which are further purified and cultured into a homogenous neural culture containing at least 50%, 55%, 60%, 65%, 70%, 75%, 80%, 85%, 90%, or 95% neurons. Methods of purifying neurons derived from human pluripotent stem cells are known in the art, for instance, see Yuan et al. (2011, PLoS ONE, 6(3): e17540), the entire content of this article is incorporated by reference herein. The present invention also contemplates all the currently available and further developed neural purification methods including any improvements.

The homogeneous neural culture produced by the present invention is characterized by neural markers, including, but not limited to, glutamatergic, cholingeric, or GABAergic markers, such as VGLUT1, CHAT and GAD67, respectively. In certain embodiments, glia could be added to the neural culture produced by the present invention. Furthermore, the purified neurons from the homogeneous neural culture are characterized for action potentials and spontaneous currents, and further measured for key characteristics of Alzheimer's disease, including, but not limited to measurements of proteolytic processing of one or amyloid precursor protein, phosphorylation of tau protein, activation of GSk3 kinase, measurement of synaptic phenotype, autophagy, or other disease behaviors.

In certain embodiments, neurons from both APP^{Dp} patients and one sAD patient exhibited significantly higher levels of secreted Aβ¹⁻⁴⁰, phospho-tau^{Thr231} (pTau) and active GSK3β (aGSK3β), relative to control neurons. Treatment of APP^{Dp} neurons with β-secretase inhibitors, but not γ-secretase inhibitors, caused significant reductions in pTau and aGSK3β levels. The present invention, thus, provides a direct relationship between APP proteolytic processing and tau phosphorylation in human neurons. Additionally, the present invention also provides that neurons with the genome of one sAD patient exhibited the phenotypes seen in familial AD samples. Therefore, using the purified human neurons obtained from the present invention, it demonstrates that (1) iPSC technology can be used to observe phenotypes of patients with Alzheimer's disease, (2) there is a causative relationship between APP processing and tau phosphorylation, and (3) neurons with the genome of an sAD patient exhibit any of the same phenotypes seen in fAD samples.

Furthermore, the present invention provides a method for diagnosing, prognosing and predicting a likelihood of development of Alzheimer's disease, and particularly for predicting a sporadic Alzheimer's disease, using the invention human neuronal model for measuring the key behaviors from the purified neurons carrying the genome of the Alzheimer's disease patients. Any known or later developed diagnostic or prognostic methods using the invention human neural models for any type of Alzheimer's disease are contemplated within the scope of the present invention. In these in vitro diagonistic or prognostic methods, the biological sample tested may be any sample of biological fluid or a tissue sample obtained by invasive or non-invasive methods.

In certain embodiments, the invention human neural models of the present invention also can be used as a method or a research tool for identifying compounds which are therapeutic candidate for treatment, diagnosis, prognosis or prevention of Alzheimer's disease. As used herein, the therapeutic candidates are compounds of any type. They may be of natural origin or may have been produced by chemical synthesis. They may be a library of structurally defined chemical compounds, of non-characterized compounds or substances, or a mixture of compounds. Various techniques can be used to screen and test these compounds and to identify the compounds of therapeutic interest.

In summary, the present invention provides a human neuronal model for hereditary and sporadic Alzheimer's disease. The human neuronal model of the present invention can be used to diagnose whether an individual is likely to develop Alzheimer's disease, either hereditary or sporadic, based on the behavior of purified neurons carrying their genome. The human neuronal model of the present invention can further be used to test candidate drugs for a therapeutic treatment of the Alzheimer's disease using human materials with disease behavior.

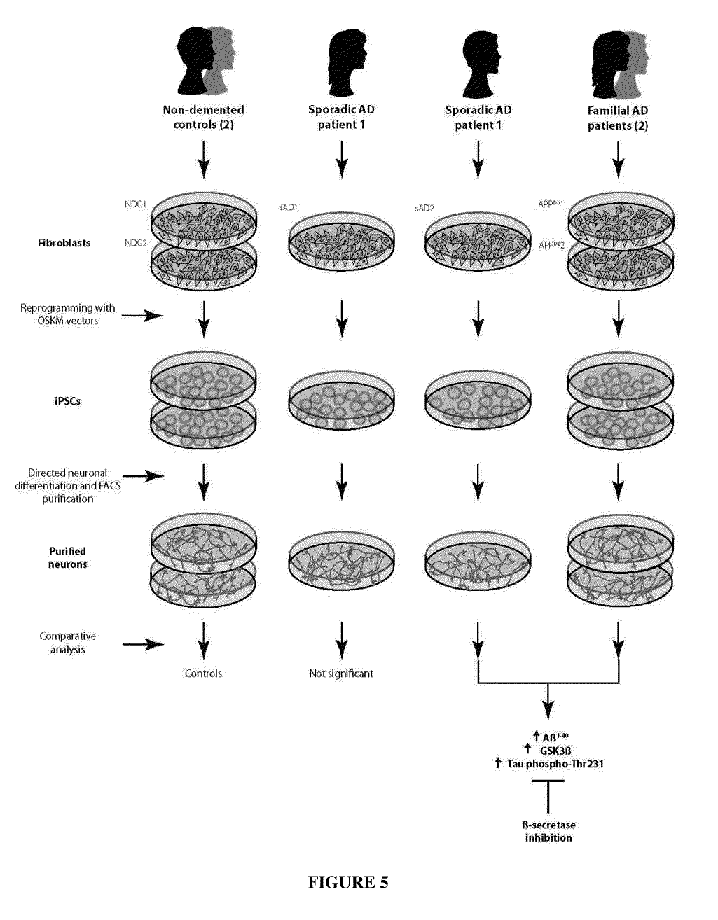

The experimental approach and findings of the present invention are summarized in FIG. 5 and described in detail below. The invention is further illustrated by the following examples and/or methods, which are not to be construed in any way as imposing limitations upon the scope thereof. It is apparent for skill artisans that various modifications and changes are possible and are contemplated within the scope of the current invention.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Various publications, including patents, published applications, technical articles and scholarly articles are cited throughout the specification. Each of these cited publications is incorporated by reference herein, in its entirety.

### EXAMPLES

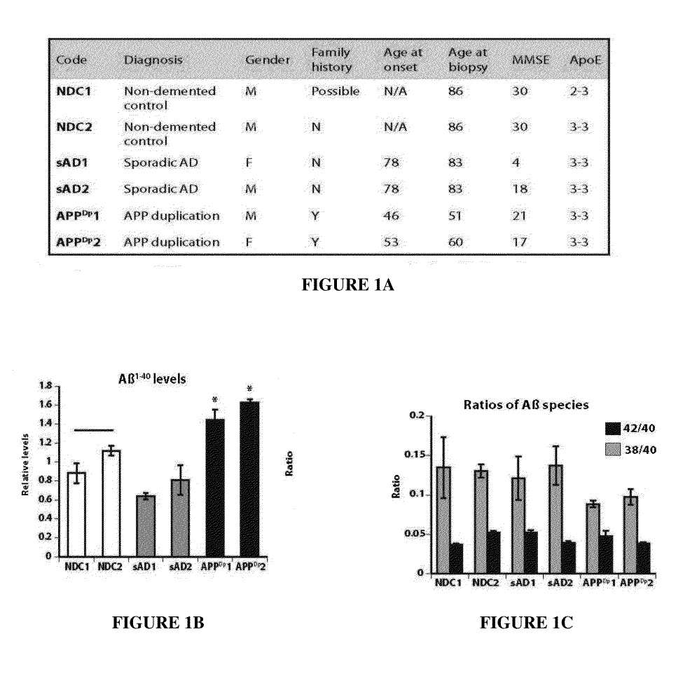

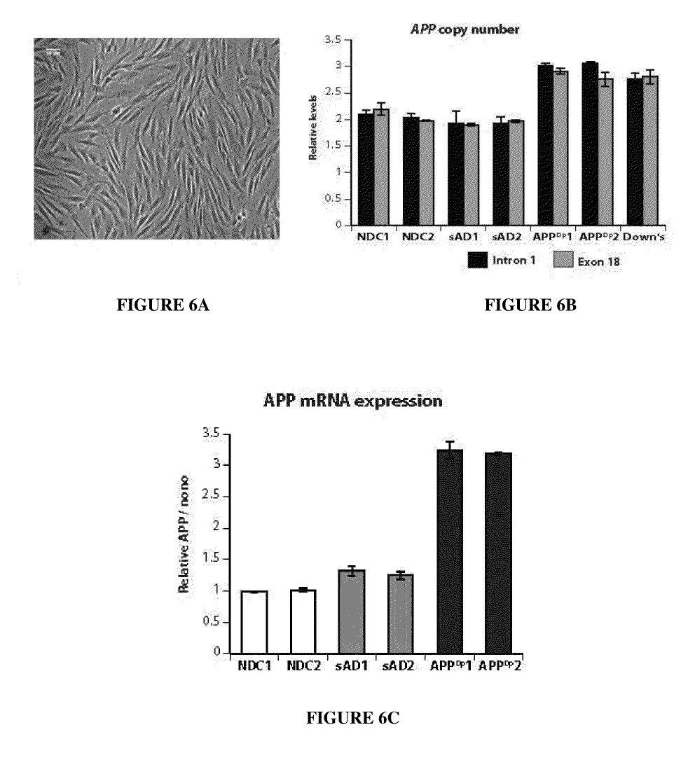

Basic fibroblast phenotypes were characterized prior to reprogramming to iPSC (FIGS. 1 and 6). APP expression and Aβ secretion were quantified in early-passage primary fibroblasts from two non-demented control individuals (NDC), two patients with sAD, and two APP^{Dp} patients. The presence of the genomic duplication was confirmed in fibroblasts. Relative to NDC and sAD cells, APP^{Dp} fibroblasts expressed higher levels of APP mRNA and secreted 1.5- to 2-fold higher amounts of Aβ¹⁻⁴⁰ peptides into the conditioned media compared to NDC cells. No significant difference was detected in Aβ 1-42/1-40 or 1-38/1-40 between any of the patients.

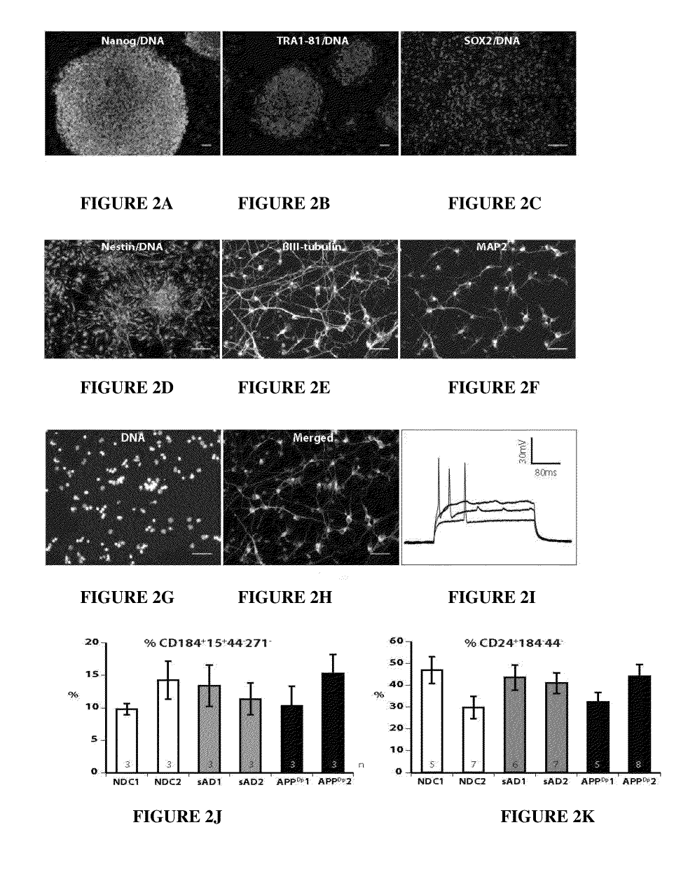

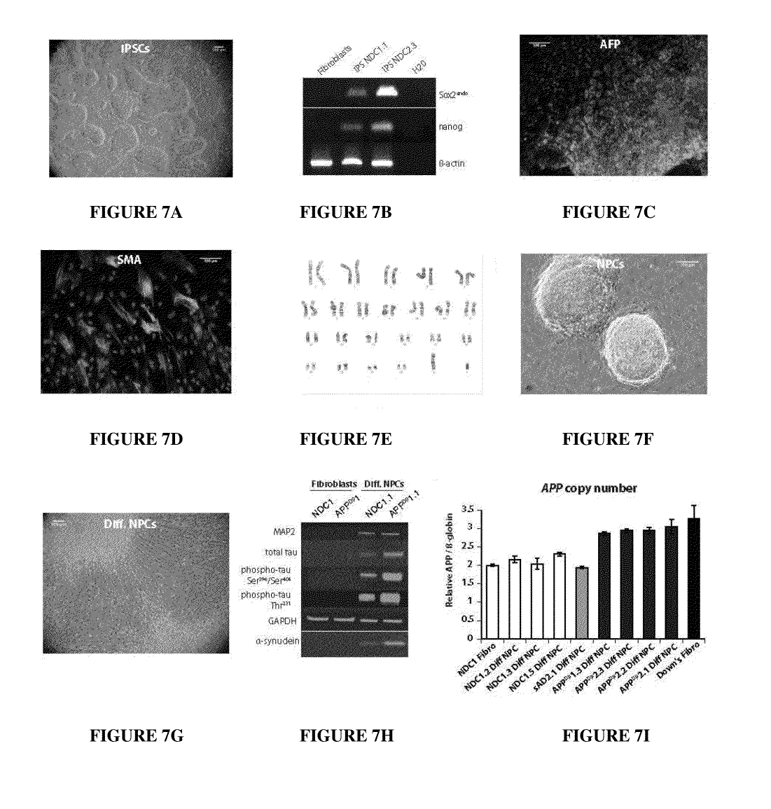

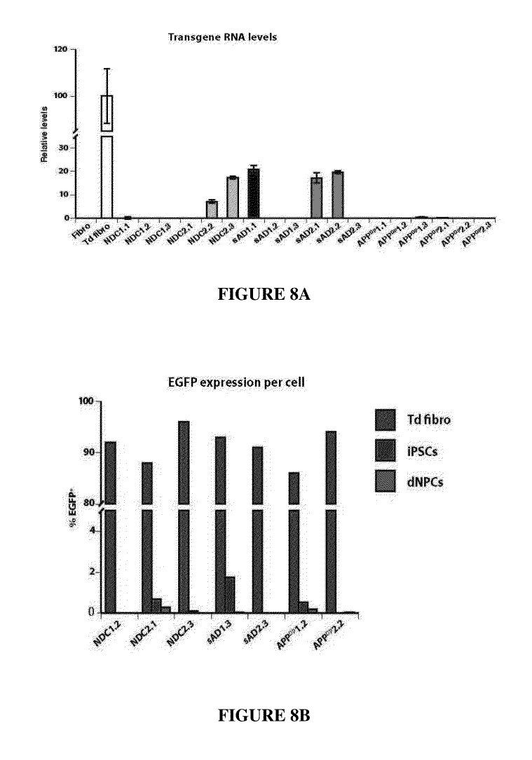

iPSC lines were generated by transducing fibroblasts with retroviruses encoding OCT4, SOX2, KLF4, c-MYC, and, in ⅓ of cases, EGFP. Each of the six individuals was represented by three clonal iPSC lines. All 18 iPSC lines maintained embryonic stem cell (ES)-like morphology and expressed the pluripotency-associated proteins nanog and TRA1-81 (FIG. 2a-b). Additionally, all lines were euploid, expressed endogenous locus-derived SOX2, silenced transgenes, and could differentiate into cells of ectodermal, mesodermal and endodermal lineages (FIG. 7a-e, and FIG. 8).

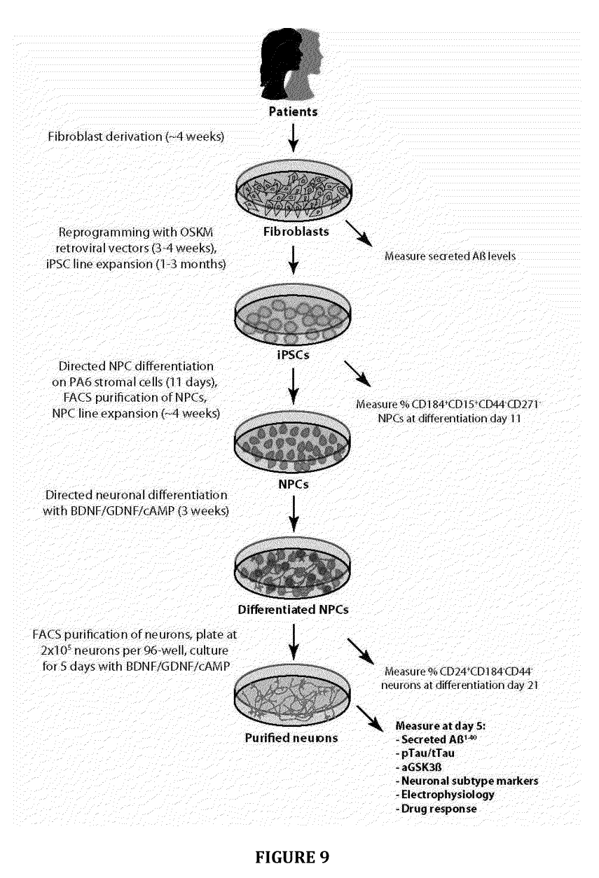

Variability in differentiation efficiency exists between pluripotent cell lines¹⁵. To analyze this variability in the iPSC lines, a FACS-based method of neuronal differentiation and purification¹⁶ (summarized in FIG. 9) were employed. This strategy allows comparison of differentiation efficiencies between patients and between clonal iPSC lines from the same patient, while simultaneously purifying neural precursor cells (NPCs) or neurons from heterogeneous cultures. Briefly, the 18 iPSC lines were first differentiated into cultures containing neural rosettes (FIG. 2f). From these cultures, NPCs were purified and the efficiency of NPC formation was assessed by CD184⁺CD15⁺CD44⁻CD271⁻ immunoreactivity. These FACS-purified NPCs maintained expression of NPC-associated markers, such as SOX2 and nestin, over multiple passages (FIG. 2c-d).

NPCs were differentiated for 3 weeks into heterogeneous cultures containing neurons (FIG. 2g-h). APP copy number was faithfully maintained in differentiated cultures (FIG. 2i). From these cultures, neurons were purified to near homogeneity, and the efficiency of neuron generation was determined by measuring CD24⁺CD184⁻CD44⁻ immunoreactivity. No significant differences between any of the individuals in the efficiency of NPC or neuronal differentiation were detected (P=0.63 and 0.14) (FIG. 2j-k). Although variability in differentiation between lines from each individual was present, differences between lines from different individuals were not greater than between lines within individuals. These results indicate that any observed biochemical alterations in neurons, if present in multiple lines from the same patient, were caused by features of that patient's genotype.

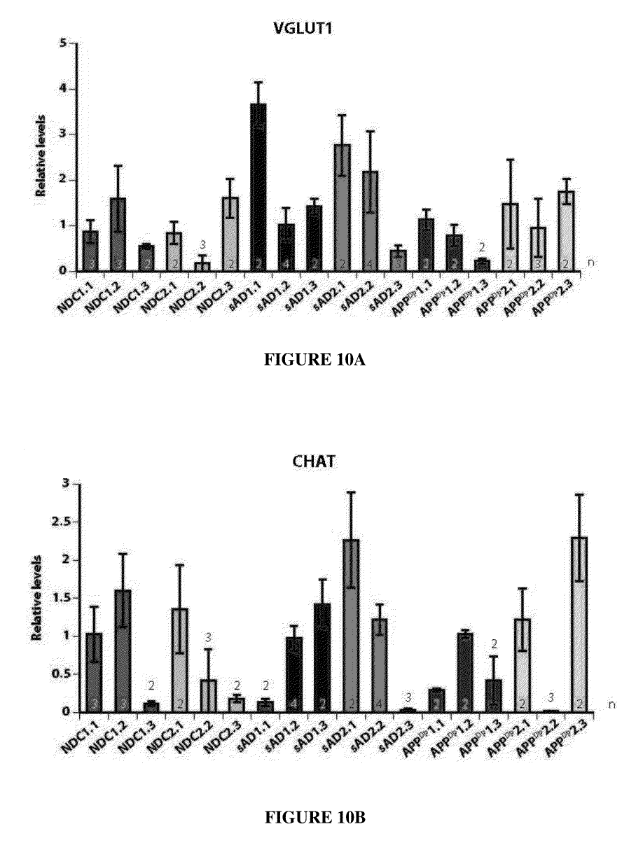

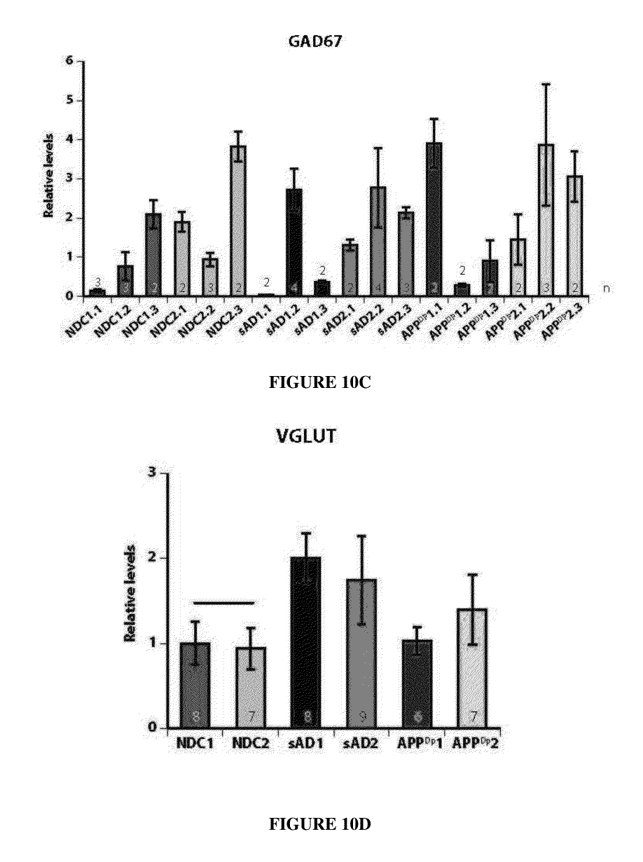

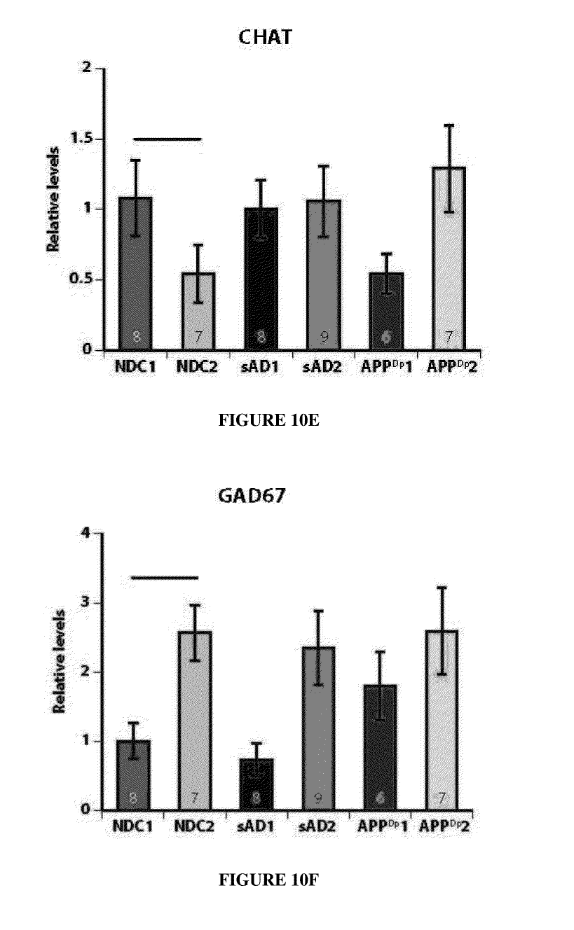

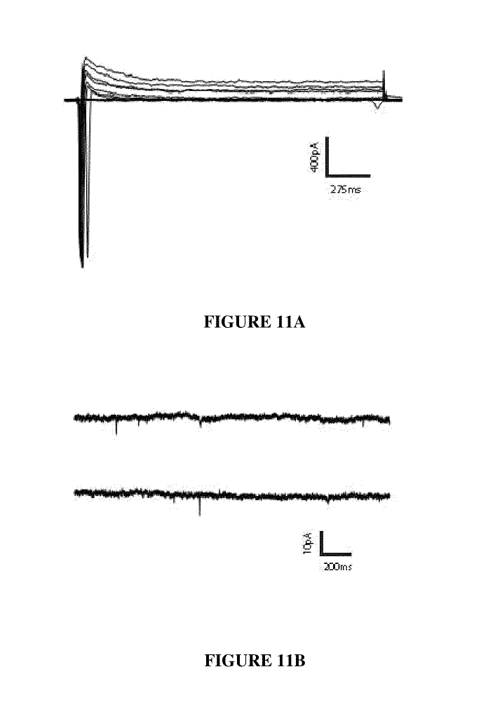

Purified neurons were plated at a density of 2×10⁵ cells per well of a 96-well plate and cultured for an additional 5 days. More than 90% of the cells in these cultures were neurons, as judged by the presence of cells possessing βIII-tubulin⁺, MAP2⁺ projections (FIG. 2e-h). No signal was detected when cultures were stained with GFAP antibody, suggesting the absence of astrocytes (data not shown). Purified neuronal cultures expressed the glutamatergic, cholingeric and GABAergic markers VGLUT1, CHAT and GAD67, respectively. No significant difference in the expression of these markers was detected between individuals by quantitative PCR (FIG. 10). Additionally, neurons were capable of generating action potentials and spontaneous currents (FIG. 2i and FIG. 11).

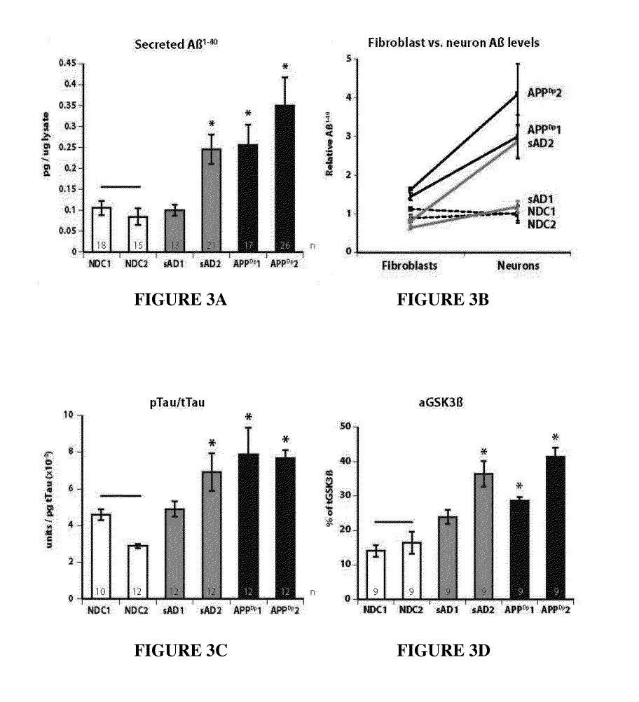

Elevated or altered secretion of Aβ peptides by fibroblasts is a feature common to all fAD mutations to date^{17,18}. It is not known if iPSC-derived neurons from fAD maintain the elevated AB production seen in the parental fibroblasts. In sAD fibroblasts and other peripheral cells, APP expression and Aβ secretion are not consistently altered¹⁹. To determine if iPSC-derived neurons from APP^{Dp} and sAD patients exhibit elevated Aβ secretion, Aβ levels in neuron-conditioned media were measured and normalized to total protein levels of cell lysates. Purified neurons from patients APP^{Dp}1 and APP^{Dp}2, each represented by three independently derived iPSC lines, secreted significantly higher levels of Aβ¹⁻⁴⁰ compared to mean NDC levels (FIG. 3a). Neurons from patient sAD2 also had significantly higher Aβ¹⁻⁴⁰ levels compared to NDC neurons, even though no difference was observed between the fibroblasts of sAD2 and NDC individuals. It was found that Aβ¹⁻⁴² and Aβ¹⁻³⁸ levels in these purified neuronal cultures were often below the detection range of the assay, owing to the relatively small number of neurons purified. By cell type, neurons exhibited a larger difference between APP^{Dp} and NDC when Aβ levels were compared to fibroblasts, suggesting that fibroblasts are not fully predictive of neuronal phenotypes (FIG. 3b).

Genetic evidence implicates altered or elevated APP processing and Aβ levels as the driving agent behind fAD⁴ and, because of identical neuropathology, sAD. However, tau, although not genetically linked to AD, forms NFTs, which correlate better with disease severity than plaque numbers²⁰. The mechanism by which altered APP processing might cause elevated pTau and NFT pathology is unclear. Tau phosphorylation at Thr231, one of several tau phosphoepitopes, regulates microtubule stability²¹ and correlates with both neurofibrillary tangle number and degree of cognitive decline^{22,23}. To determine if tau phosphorylation at Thr231 is elevated in APP^{Dp} and sAD neurons, the amount of phospho-tau^{Thr231} relative to total tau levels (tTau) was measured in lysates from purified neurons from three iPSC lines from each of the NDC, sAD and APP^{Dp} patients. Neurons from both APP^{Dp} patients had significantly higher pTau/tTau than neurons from NDC lines (FIG. 3c). pTau/tTau in the two sAD patients mirrored the Aβ findings: No difference was observed between sAD1 and NDC neurons while sAD2 neurons had significantly increased pTau/tTau.

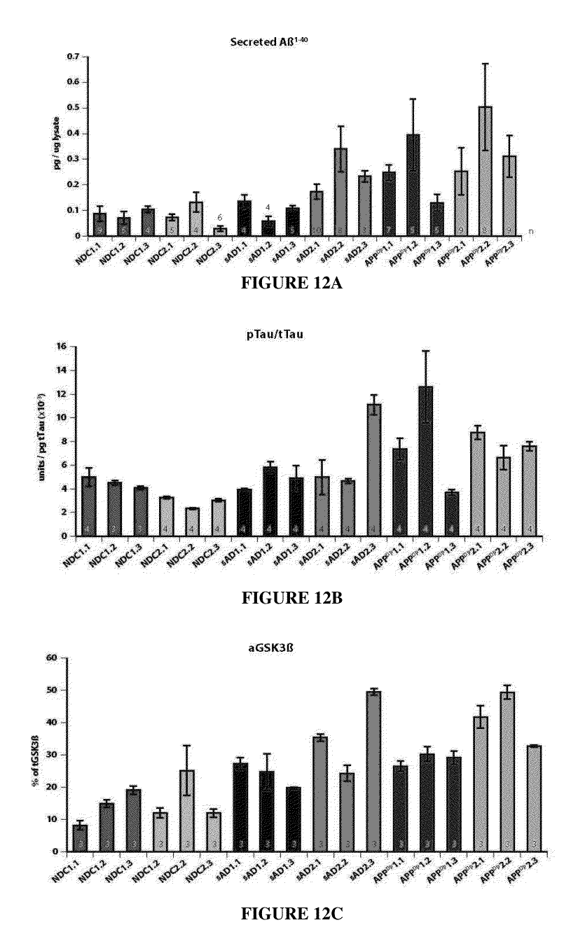

Tau can be phosphorylated by multiple kinases. The kinase GSK3β (also known as tau protein kinase I) can phosphorylate tau at Thr231 in vitro and co-localizes with NFTs and pre-tangle phosphorylated tau in sAD postmortem neurons. GSK3β is thought to be constitutively active but is inactivated when phosphorylated at Ser9²⁵. To determine if iPSC-derived neurons with elevated pTau have increased GSK3β activity, the proportion of active GSK3β (aGSK3β) in purified neurons was calculated by measuring the amount of GSK3β lacking phosphorylation at Ser9 relative to total GSK3β levels. It was found that neurons from patients APP^{Dp}1, APP^{Dp}2 and sAD2 had significantly higher aGSK3β than NDC neurons (FIG. 3d). Aβ, pTau/tTau, aGSK3β, and other measurements are summarized for each iPSC line are shown in FIG. 12.

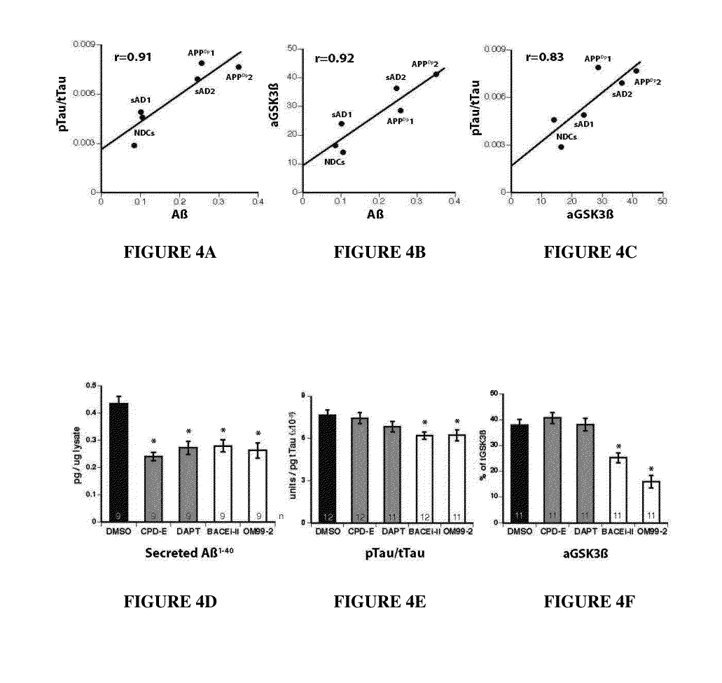

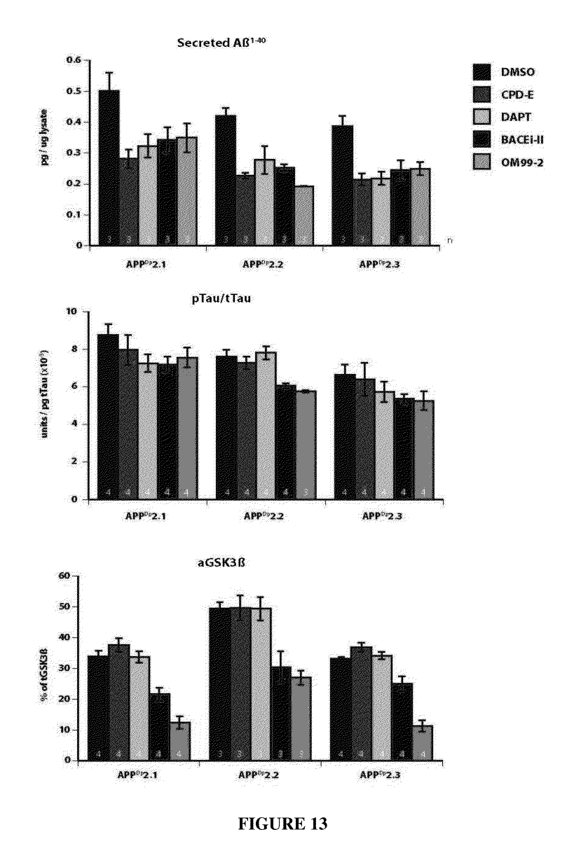

Although Aβ, pTau and GSK3β clearly play roles in AD pathogenesis, their relationship is unknown. The iPSC-derived neurons were found to exhibit strong correlations between Aβ¹⁻⁴⁰, pTau/tTau and aGSK3β levels (FIG. 4a-c). In contrast, correlation coefficients between these measurements and neuronal subtype markers, tTau, total protein levels or transgene expression levels were much weaker, with the exception of aGSK3β and tTau (Table 2b). If APP proteolytic products, such as Aβ, play a causative role in pTau and aGSK3β elevation, then inhibiting γ- and β-secretase activity should reduce pTau and aGSK3β. To determine whether inhibiting γ- and β-secretase activity reduces pTau and aGSK3β, purified neurons from patient APP^{Dp}2's three iPSC lines with We treated γ-secretase inhibitors (200 nM CPD-E or 200 nM DAPT) and β-secretase inhibitors (10 μM BACEi-II or 750 nM OM99-2) for 24 hours and measured Aβ, pTau/tTau and aGSK3β levels compared to vehicle-treated samples. All inhibitors reduced Aβ¹⁻⁴⁰ by 36-45% (FIG. 4d). It was found that, while neither γ-secretase inhibitor significantly differed from control samples, both β-inhibitors significantly reduced pTau/tTau and aGSK3β levels (FIG. 4e-f, and shown per iPSC line in FIG. 13).

Thus, relative to two NDC patients, significant differences were observed in (1) Aβ¹⁻⁴⁰ levels, (2) phospho-tau, and (3) GSK3β activity in purified neurons from two APP^{Dp} patients, each represented by three clonally derived iPSC lines. Not only did strong correlations exist between Aβ¹⁻⁴⁰, pTau/tTau and aGSK3β, but both pTau/tTau and aGSK3β were significantly reduced in APP^{Dp} neurons following treatment with β-secretase inhibitors, suggesting that the APP processing pathway plays a causative role in tau Thr231 phosphorylation in human neurons. Interestingly, increased pTau has been observed to destabilize microtubules^{5,21}, so the observation that pTau was elevated in APP^{Dp} neurons is in agreement with a previous report that mouse models harboring APP duplications have axonal phenotypes, such as reduced axonal transport²⁶.

Additionally, these murine axonal phenotypes appeared to correlate better with levels of β-secretase products than γ-secretase products. The secretase inhibitor findings are further mirrored in a report showing that endosomal pathology in human Down's syndrome fibroblasts is reduced by lowering BACE expression levels but not significantly affected by γ-secretase inhibition²⁷. Because of GSK3β's known role as a tau kinase, these findings that GSK3β was altered in APP^{Dp} neurons and could be rescued by β-secretase inhibition provide additional evidence that GSK3β plays a role in the relationship between Aβ and pTau.

Similar to neurons from APP^{Dp} patients, neurons from patient sAD2 exhibited increased levels of Aβ¹⁻⁴⁰ and pTau relative to NDC neurons. These aberrations were not observed in the parental fibroblasts, suggesting a cell type-specific phenotype. The specific cell types that possess this phenotype can be further investigated by measuring Aβ levels following the differentiation of iPSC into various non-ectodermal and alternate ectodermal lineages. No aberrant Aβ or pTau levels was observed in patient sAD1, which raises the question of what percentage of sAD patients will resemble sAD2, in terms of displaying biochemical alterations in iPSC-derived neurons. This observation may also indicate that Alzheimer's disease might be sub-divided depending on whether neurons themselves are altered as opposed to other cell types. Examining larger numbers of patients and controls provides great insight into the mechanisms behind the observed heterogeneity in sAD pathogenesis, the role of different cell types, patient-specific drug responses, and prospective diagnostics. Nevertheless, the present invention provides an opportunity for the genetic dissection of phenotypes observed in neurons from patient sAD2.

In summary, the present invention provides that iPSC technology can be used in concert with postmortem samples and animal models to study early Alzheimer's disease pathogenesis and drug response in sAD and fAD. Any cell type can be differentiated and purified from iPSC, which creates new ways to study cell autonomous versus non-cell autonomous disease mechanisms. Many pathologies shown to be associated with the earliest stages of Alzheimer's disease, such as aberrant axonal transport²⁸ and endocytic activity²⁹, are best studied in live cells, and iPSC-derived neural cells are unique tools that can be used to further elucidate the cascade of events that drive Alzheimer's disease.

### Methods Summary

### Patients and Fibroblast Derivation

NDC and sAD individuals were enrolled in the longitudinal study at the UCSD Alzheimer's Disease Research Center. APP^{Dp} individuals were patients at the Department of Clinical Medicine, Neurology, Oulu University Hospital, Oulu, Finland. For all individuals, dermal punch biopsies were taken following informed consent and IRB approval. Primary fibroblast cultures were established from biopsies using established methods³⁰. Fibroblasts were cultured in DMEM containing 15% FBS, L-glutamine, and Penicillin/Streptomycin.

iPSC Generation, Differentiation, and Expansion

Primary fibroblast cultures were established from dermal punch biopsies taken from individuals following informed consent and IRB approval. To generate iPSCs, fibroblasts were transduced with retroviruses containing the cDNAs for OCT4, SOX2, KLF4, c-MYC and EGFP and seeded into human ES cell culture conditions. Differentiation to NPCs and neurons followed the protocol of Yuan, et al¹⁶.

iPSC were generated as described³¹, with the following modifications. The cDNAs for OCT4, SOX2, KLF4, c-MYC and EGFP were cloned into pCX4 vectors³² and vectors were packaged into VSVG-pseudotyped retroviruses. For each patient, 3 independent viral transductions were performed. Three wells each containing ˜1×10⁵ fibroblasts were transduced with retroviruses. On days 2-8, 2 mM valproic acid was added to cultures. Potential iPSC colonies were picked at ˜3 weeks and transferred to 96-well plates containing irradiated mouse embryonic fibroblasts (MEFs). For passaging, cells were dissociated with TrypLE (Invitrogen). Efficiency of potential iPSC colony formation was roughly 100 colonies per 1×10⁵ fibroblasts at 3 weeks. Efficiency of successful establishment of a stable iPSC line from an initial colony was roughly 10%.

### Karyotype Analysis and Endoderm/Mesoderm Generation

Karyotype analysis was performed by Cell Line Genetics (Madison, Wis.). To determine if iPSC lines could generate endoderm and mesoderm, iPSC cultures were dissociated with dispase, and embryoid bodies were generated by plating cultures in low-attachment culture plates in media containing 15% fetal bovine serum (FBS). After 7 days, cultures were plated on Matrigel (BD Biosciences)—coated glass coverslips and cultures for an additional 7 days. At this point cultures were harvested for immunocytochemistry.

### Genotyping and RNA Expression

To determine APP copy number, genomic DNA was isolated from fibroblasts or NPCs differentiated for 3 weeks. Quantitative PCR (QPCR) was performed using FastStart Universal SYBR Green Master Mix (Roche) and primers that amplify APP intron 1, APP exon 18, β-globin, and albumin (primer sequences available on request). Reactions were performed and analyzed on an Applied Biosystems 7300 Real Time PCR System using the ΔΔCt method. APP levels were normalized to mean β-globinlalbumin. Results are expressed relative to NDC1. To compare RNA levels between samples, RNA was purified (PARIS kit, Ambion), DNase treated (Ambion) and reverse transcribed (Superscript II, Invitrogen). QPCR was performed using the reagents described above with primers that recognize all APP isoforms. PCR to detect endogenous SOX2 expression was performed using Qiagen HotStarTaq and primers previously described³³.

### Immunocytochemistry and FACS

Cells were fixed in 4% paraformaldehyde, permeabilized with buffer containing TritonX-100, and stained with primary and secondary antibodies (see below). Samples were imaged on a Nikon TE2000-U inverted microscope and acquired using Metamorph software (Molecular Devices). ImageJ software (NIH) was used to pseudo-color images, adjust contrast, and add scale bars. FACS experiments were carried out on a FACS Aria II cytometer (BD Biosciences) and analyzed using FloJo software (Tree Star).

### Antibodies

The antibodies used for FACS purification of cells were CD184-APC, CD15-FITC, CD24-PECy7, CD44-PE, and CD271-PE (all from BD Biosciences) and were used at a concentration of 1 test per 1×10⁶ cells. The following antibodies were from Millipore: SOX1 (1:2000), SOX2 (1:2000), MAP2a/b (1:500), and APP^{FL} (22C11, 1:1000). Other antibodies and dilutions: α-tubulin (Sigma 1:250k), APP^{CTF} (Zymed 1:500), Tau^{PHF1} (Pierce 1:500), nanog (Santa-Cruz 1:200), nestin (Santa-Cruz 1:200), βIII-tubulin (Covance 1:500), GFAP (Sigma 1:200), PAX-6 (Developmental Studies Hybridoma Bank 1:2000), anti-rabbit Alexa Fluor 488 (Invitrogen 1:200), and anti-rabbit Alexa Fluor 568 (Invitrogen 1:200).

### Neuronal Differentiation and Culture

Differentiation to NPCs and neurons followed Yuan, et al³⁴. Differentiation began with a confluent 10 cm dish of undifferentiated iPSC. Cells were dissociated with Accutase (Innovative Cell Technologies) and for each plate, ˜5×10⁵ TRA1-81⁺ iPSC were isolated by FACS. 3×10⁵ FACS-purified cells were seeded onto 3×10 cm plates that were seeded the previous day with 5×10⁵ PA6 cells³⁵. At day 11, cells were dissociated with Accutase and ˜5×10⁵ CD184⁺CD15⁺CD44⁻CD271⁻ NPCs were FACS-purified and plated onto poly-ornithine/laminin-coated plates and cultured with bFGF. On passage 7, NPCs were differentiated with BDNF, GDNF and cAMP. After 3 weeks of differentiation, cells were dissociated with Accutase and 1-2 million CD24⁺CD184⁻CD44⁻ cells were collected and carefully plated at a density of 2×10⁵ per 96-well. Cells were cultured for an additional 5 days with a full-media change on day 3. On day 5, conditioned media were harvested for AB analysis and cells were harvested for MSD, immunocytochemical or electrophysiological analysis. Differentiation methods are additionally summarized in FIG. 9.

Neurons were FACS-purified from NPCs differentiated for 3 weeks and cultured for an additional 5 days. At this point, conditioned media were harvested and cultures were lysed. Aβ, pTau/tTau and aGSK3β levels were measured by multi-spot electrochemiluminescence assays (Meso Scale Diagnostics).

### Electrophysiology Methods

Whole-cell perforated patch recordings were performed on purified neurons cultured for 5 days. Methods were previously described.³⁶

Aβ, pTau/tTau and aGSK3β measurements

AB was measured with MSD Human (6E10) Abeta3-Plex Kits (Meso Scale Discovery). pTau/tTau was measured with a MSD Phospho(Thr231)/Total Tau Kit. aGSK3β was measured with MSD Phospho/Total GSK-3b Duplex Kit. For these assays, each patient was represented by 3 iPSC lines, and for each iPSC line, multiple biological replicates were studied. For MSD assays a standard curve was generated and only samples that fell on the linear range were analyzed. Fibroblast and neuronal Aβ levels were normalized to total protein levels determined by BCA assay (Thermo Scientific). pTau/tTau was determined by dividing the calculated concentration of pTau by the calculated concentration of tTau. aGSK3β (the percent of unphosphorylated GSK3β at Ser9) was calculated by manufacturer's recommendations: [1−(2*phospho signal)/(phospho signal+total signal)]*100.

### Inhibitor Treatments

CPD-E (Compound-E) and DAPT were used at a final concentration of 200 nM. BACEi-II and OM99-2 were used at 10 μM and 750 nM, respectively. One μL of inhibitor or vehicle was added to the existing culture media of parallel cultures on day 4 and cultures were harvested on day 5. All inhibitors were from EMD Chemicals.

### Statistics

Data were analyzed using JMP software (SAS Institute). P<0.05 was considered statistically significant. Bar graphs display mean ±s.e.m. Comparisons between individuals were made by performing ANOVA followed by Tukey's HSD posthoc test. All biological replicates for each individual were compared to the pool of all NDC replicates. Drug responses were compared to controls by Dunnett's method. Correlations were determined by calculating Pearson coefficients (r).

P<0.05 was considered statistically significant. Bar graphs display mean ±s.e.m. Individuals were statistically compared to the total NDC pool by Tukey's HSD test. Drug responses were compared to controls by Dunnett's method.

### REFERENCES

1 Remes, A. M. et al. Hereditary dementia with intracerebral hemorrhages and cerebral amyloid angiopathy. Neurology 63, 234-240 (2004).
2 Rovelet-Lecrux, A. et al. APP locus duplication in a Finnish family with dementia and intracerebral haemorrhage. J. Neurol. Neurosurg. Psychiatry 78, 1158-1159 (2007).
3 Alzheimer, A. Ueber eine eigenartige Erkrankung der Himrinde. Allg. Z Psychiat. Med. 64, 146-148 (1907).
4 Tanzi, R. E. & Bertram, L. Twenty Years of the Alzheimer's Disease Amyloid Hypothesis: A Genetic Perspective. Cell 120, 545-555 (2005).
5 Ballatore, C., Lee, V. M. Y. & Trojanowski, J. Q. Tau-mediated neurodegeneration in Alzheimer's disease and related disorders. Nat. Rev. Neurosci., 663-672 (2007).
6 Gatz, M. et al. Role of Genes and Environments for Explaining Alzheimer Disease. Arch. Gen. Psychiatry 63, 168-174 (2006).
7 Games, D. et al. Alzheimer-type neuropathology in transgenic mice overexpressing V717F β-Amyloid precursor protein. Nature 373, 523-527 (1995).
8 Roberson, E. D. et al. Reducing Endogenous Tau Ameliorates Amyloid β-Induced Deficits in an Alzheimer's Disease Mouse Model. Science 316, 750-754 (2007).
9 Busciglio, J., Lorenzo, A., Yeh, J. & Yankner, B. A. β-amyloid fibrils induce tau phosphorylation and loss of microtubule binding. Neuron 14, 879-888 (1995).
10 Takahashi, K. & Yamanaka, S. Induction of Pluripotent Stem Cells from Mouse Embryonic and Adult Fibroblast Cultures by Defined Factors. Cell 126, 663-676 (2006).
11 Ebert, A. D. et al. Induced pluripotent stem cells from a spinal muscular atrophy patient. Nature 457, 277-280 (2009).
12 Lee, G. et al. Modelling pathogenesis and treatment of familial dysautonomia using patient-specific iPSCs. Nature 461, 402-406 (2009).
13 Marchetto, M. C. N. et al. A Model for Neural Development and Treatment of Rett Syndrome Using Human Induced Pluripotent Stem Cells. Cell 143, 527-539 (2010).
14 Urbach, A., Bar-Nur, O., Daley, G. Q. & Benvenisty, N. Differential Modeling of Fragile X Syndrome by Human Embryonic Stem Cells and Induced Pluripotent Stem Cells. Cell Stem Cell 6, 407-411 (2010).
15 Osafune, K. et al. Marked differences in differentiation propensity among human embryonic stem cell lines. Nat. Biotechnol. 26, 313-315 (2008).
16 Yuan, S. et al. Cell-surface marker signatures for the isolation of neural stem cells, glia and neurons derived from human pluripotent stem cells. PLoS One (Submitted).
17 Citron, M. et al. Excessive production of amyloid β-protein by peripheral cells of symptomatic and presymptomatic patients carrying the Swedish familial Alzheimer disease mutation. Proc. Natl. Acad. Sci. U.S.A. 91, 11993-11997 (1994).
18 Scheuner, D. et al. Secreted amyloid β-protein similar to that in the senile plaques of Alzheimer's disease is increased in vivo by the presenilin 1 and 2 and APP mutations linked to familial Alzheimer's disease. Nat. Med. 2, 864-870 (1996).
19 Gasparini, L. et al. Peripheral markers in testing pathophysiological hypotheses and diagnosing Alzheimer's disease. FASEB J. 12, 17-34 (1998).
20 Arriagada, P. V., Growdon, J. H., Hedley-Whyte, E. T. & Hyman, B. T. Neurofibrillary tangles but not senile plaques parallel duration and severity of Alzheimer's disease. Neurology 42, 631 (1992).
21 Cho, J.-H. & Johnson, G. V. W. Primed phosphorylation of tau at Thr231 by glycogen synthase kinase 3B (GSK313) plays a critical role in regulating tau's ability to bind and stabilize microtubules. J. Neurochem. 88, 349-358 (2004).
22 Buerger, K. et al. CSF tau protein phosphorylated at threonine 231 correlates with cognitive decline in MCI subjects. Neurology 59, 627-629 (2002).
23 Buerger, K. et al. CSF phosphorylated tau protein correlates with neocortical neurofibrillary pathology in Alzheimer's disease. Brain 129, 3035-3041 (2006).
24 Cho, J. & Johnson, G. Glycogen Synthase Kinase 3B Phosphorylates Tau at Both Primed and Unprimed Sites. J. Biol. Chem. 278, 187-193 (2003).
25 Dajani, R. et al. Crystal Structure of Glycogen Synthase Kinase 3B: Structural Basis for Phosphate-Primed Substrate Specificity and Autoinhibition. Cell 105, 721-732 (2001).
26 Salehi, A. et al. Increased App Expression in a Mouse Model of Down's Syndrome Disrupts NGF Transport and Causes Cholinergic Neuron Degeneration. Neuron 51, 29-42 (2006).
27 Jiang, Y. et al. Alzheimer's-related endosome dysfunction in Down syndrome is Aβ-independent but requires APP and is reversed by BACE-1 inhibition. Proc. Natl. Acad. Sci. U.S.A. 107, 1630-1635 (2010).
28 Stokin, G. B. et al. Axonopathy and Transport Deficits Early in the Pathogenesis of Alzheimer's Disease. Science 307, 1282-1288 (2005).
29 Cataldo, A., Barnett, J., Pieroni, C. & Nixon, R. Increased Neuronal Endocytosis and Protease Delivery to Early Endosomes in Sporadic Alzheimer's Disease: Neuropathologic Evidence for a Mechanism of Increased β-Amyloidogenesis. J. Neurosci. 17, 6142-6151 (1997).
30 Takashima, A. in Current Protocols in Cell Biology Ch. 2, 12 (John Wiley & Sons, 2001).
31 Park, I.-H., Lerou, P. H., Zhao, R., Huo, H. & Daley, G. Q. Generation of human-induced pluripotent stem cells. Nat. Protoc. 3, 1180-1186 (2008).
32 Akagi, T., Sasai, K. & Hanafusa, H. Refractory nature of normal human diploid fibroblasts with respect to oncogene-mediated transformation. Proc. Natl. Acad. Sci. U.S.A. 100, 13567-13572 (2003).
33 Takahashi, K. et al. Induction of Pluripotent Stem Cells from Adult Human Fibroblasts by Defined Factors. Cell 131, 861-872 (2007).
34 Yuan, S. et al. Cell-surface marker signatures for the isolation of neural stem cells, glia and neurons derived from human pluripotent stem cells. PLoS One (Submitted).
35 Kawasaki, H. et al. Induction of Midbrain Dopaminergic Neurons from ES Cells by Stromal Cell Derived Inducing Activity. Neuron 28, 31-40 (2000).
36 Marchetto, M. C. N. et al. A Model for Neural Development and Treatment of Rett Syndrome Using Human Induced Pluripotent Stem Cells. Cell 143, 527-539 (2010).

What is claimed is:

1. A human neuronal model comprising human neural cells derived from human induced pluripotent stem cells (iPSCs) from a patient with Alzheimer's disease.

2. The human neuronal model of claim 1, wherein said Alzheimer's disease is sporadic Alzheimer's disease.

3. The human neuronal model of claim 1, wherein said Alzheimer's disease is hereditary Alzheimer's disease.

4. The human neuronal model of claim 1, further comprising human neurons developed from said human neural cells.

5. The human neuronal model of claim 4, wherein said human neurons present key behaviors of Alzheimer's disease.

6. The human neuronal model of claim 5, wherein said key behaviors are measurements of proteolytic processing of one or more amyloid precursor protein, phosphorylation of tau protein, activation of GSk3 kinase, measurement of synaptic phenotype, autophagy, or other disease behaviors.

7. A method of making a human neuronal model of claim 1, comprising:
a) isolating fibroblasts from a patient with Alzheimer's disease;
b) reprogramming said fibroblasts to induced pluripotent stem cells (iPSCs);
c) further differentiating said iPSCs into cultures containing neural rosettes;
d) purifying neural precursor cells (NPCs);
e) further differentiating purified NPCs into heterogeneous cultures containing neurons;
f) purifying neurons from the heterogeneous cultures;
g) further culturing purified neurons into a homogeneous neural culture containing at least 90% neurons.

8. The method of claim 7, wherein said iPSCs are reprogrammed by transducting said fibroblasts with retrovirus encoding OCT4, SOX2, KLF4, c-MYC, optionally with EGFP.

9. The method of claim 7, further comprising adding glia to said cultures.

10. The method of claim 7, further comprising characterizing the neural culture for neural markers.

11. The method of claim 10, wherein said neural markers are glutamatergic, cholingeric, or GABAergic markers.

12. The method of claim 7, further comprising characterizing the purified neurons for action potentials and spontaneous currents, or measurement of key behaviors of Alzheimer's disease.

13. The method of claim 12, wherein said key behaviors are measurements of proteolytic processing of one or amyloid precursor protein, phosphorylation of tau protein, activation of GSk3 kinase, measurement of synaptic phenotype, autophagy, or other disease behaviors.

13. A method for diagnosing Alzheimer's disease using the human neuronal model of claim 1.

14. The method of claim 13 comprising diagnosing and predicting a likelihood of development of Alzheimer's disease based on the behavior of purified neurons from the human neuronal model.

15. The method of claim 13, wherein the Alzheimer's disease is sporadic Alzheimer's disease.

16. A method of identifying a candidate therapeutic agent for Alzheimer's disease treatment comprising combining the human neuronal model of claim 1 with an agent, determining therapeutically beneficial changes in the neuronal model in response to the combination with the agent, thereby identifying the agent as a candidate therapeutic agent.

17. The method of claim 16, wherein the therapeutically beneficial changes are selected from advantageous modification of the model expression profile, phenotype or morphology.
